(19) United States
(12) Patent Application Publication
(10) Pub. No.: US 2005/0176918 A1
(43) Pub. Date: Aug. 11, 2005

(54) Production of bisphenol a with reduced isomer formation

(76) Inventors: Rainer Neumann, Tolz (DE); Ulrich Blaschke, Krefeld (DE); Stefan Westernacher, Kempen (DE)

Correspondence Address: BAYER MATERIAL SCIENCE LLC, 100 BAYER ROAD, PITTSBURGH, PA, 15205, US

(21) Appl. No.: 11/043,769

(22) Filed: Jan. 26, 2005

(30) Foreign Application Priority Data
Feb. 5, 2004 (DE) 1020040057249

(51) Int. Cl. C08G 64/00; C07C 37/68

(52) U.S. Cl. 528/196; 568/426

(57) ABSTRACT

A process for the production of bisphenol A is disclosed. The process entails a) mixing phenol and acetone to form a mixture, and b) bringing the mixture to a temperature of 48 to 54° C. to form a warm mixture, and c) commencing a reaction between phenol and acetone upon contacting the warm mixture with an acid ion exchange catalyst to form a product mixture that contains bisphenol A. The bisphenol A may be extracted from the product mixture by crystallization and filtration.

### FIELD OF THE INVENTION

The invention relates to a process for the production of bisphenol A.

### TECHNICAL BACKGROUND OF THE INVENTION

Bisphenols as condensation products of phenols and carbonyl compounds are starting substances or intermediates for the production of a large number of commercial products. Of particular industrial significance is the condensation product of the reaction between phenol and acetone, 2,2-bis(4-hydroxy-phenyl)propane (bisphenol A, BPA). BPA is used as a starting substance for the production of various types of polymer materials, such as e.g. polyarylates, polyether imides, polysulfones and modified phenol-formaldehyde resins. Preferred areas of application lie in the production of epoxy resins and polycarbonates.

Industrially relevant production methods for BPA are known and are based on the acid-catalyzed reaction of phenol with acetone, wherein a phenol-acetone ratio of more than 5:1 is preferably established in the reaction. This reaction conventionally takes place continuously, and generally at temperatures of 45 to 110° C., preferably at 50 to 80° C., as described in DE-A-199 57 602. Both homogeneous and heterogeneous Bronsted or Lewis acids can be used as acid catalysts, such as, for example, strong mineral acids, e.g. hydrochloric or sulfuric acid. Gel-like or macroporous sulfonated crosslinked polystyrene resins (acid ion exchangers) are preferably used. Divinylbenzene is normally used as the crosslinking agent, but others, such as divinylbiphenyl, can also be employed. In addition to the catalyst, a co-catalyst can be used. These are usually thiols, which carry at least one SH function. The co-catalyst can be both homogeneously dissolved in the reaction solution and, in the case of the acid ion exchangers, fixed on the catalyst itself. Homogeneous co-catalysts are e.g. mercaptopropionic acid, hydrogen sulfide, alkyl sulfides, such as e.g. ethyl sulfide, and similar compounds.

Fixed co-catalysts are aminoalkyl thiols and pyridylalkyl thiols which are ionically bonded to the catalyst, it being possible for the SH function to be protected and only released during or after fixing to the catalyst. The co-catalyst can also be covalently bonded to the catalyst as alkyl or aryl thiol.

When phenol is reacted with acetone in the presence of acid catalysts, a product mixture is formed which, in addition to unreacted phenol and possibly acetone, primarily contains BPA and water. In addition, small quantities of typical by-products of the condensation reaction occur, such as e.g. 2-(4-hydroxyphenyl)-2-(2-hydroxyphenyl)propane (o,p-BPA), substituted indanes, hydroxyphenyl-indanols, hydroxyphenylchromanes, spirobisindanes, substituted indenols, substituted xanthenes and more highly condensed compounds with three or more phenyl rings in the molecular backbone. In addition, other secondary components, such as anisole, mesityl oxide, mesitylene and diacetone alcohol, can form by self-condensation of the acetone and reaction with impurities in the raw materials.

For economic and technical reasons, the reaction is usually performed in such a way that a hundred per cent conversion of the acetone is not achieved and 0.1-0.6 wt. % acetone is still contained in the reactor discharge.

The above-mentioned by-products, such as water, but also the unreacted feedstocks, such as phenol and acetone, impair the suitability of BPA for the production of polymers and have to be separated off by suitable processes. High demands are made of the purity of the raw material BPA, particularly for the production of polycarbonate.

One processing and purification method for BPA takes place by separating BPA out of the reaction mixture in the form of an approximately equimolar crystalline adduct with phenol by cooling the reaction mixture, allowing the BPA-phenol adduct to crystallise out in a suspension crystallization. The BPA-phenol adduct crystals are then separated from the liquid phase by suitable apparatus for solid-liquid separation, such as rotary filters or centrifuges, and passed on for further purification.

Adduct crystals obtained in this way typically exhibit a purity of >99 wt. % BPA, based on the sum of BPA and the secondary components, with a phenol proportion of approx. 40 wt. %. By washing with suitable solutions, which typically contain one or more components from the group consisting of acetone, water, phenol, BPA and secondary components, the adduct crystals can be freed of impurities adhering to the surface.

The liquid stream forming during the solid-liquid separation (mother liquor) contains phenol, BPA, water formed during the reaction and unreacted acetone, and is enriched in the secondary components typically forming during BPA production. This mother liquor stream is generally fed back into the reaction unit. To maintain the catalytic activity of the acid ion exchangers, water that has formed previously is removed by distillation and any acetone still present is also removed from the mother liquor at the same time. The dehydrated reaction stream thus obtained is supplemented with phenol, acetone and optionally co-catalyst and is fed back into the reaction unit. However, the phenol can also be added, wholly or partly, before the dehydration. Alternatively, water and acetone can also be removed by distillation before carrying out the suspension crystallization of the BPA-phenol adduct. In the distillation steps mentioned, a partial quantity of the phenol present in the reaction solution can also be removed by distillation at the same time.

In a circulating operation of this type, the problem occurs that by-products from BPA production become concentrated in the circulating stream and lead to the deactivation of the catalyst system and to poorer product qualities. To avoid excessive concentration of secondary components in the circulating stream, a partial quantity of the circulating stream—optionally after partial or complete recovery of phenol by distillation—is discharged from the process chain as so-called BPA resin.

Furthermore, part or all of the circulating stream can be passed through a rearrangement unit filled with acid ion exchanger after the solid-liquid separation and before or after the separation of water and residual acetone. This unit is generally operated at higher temperatures than the reaction unit. In this rearrangement unit, some of the secondary components from BPA production present in the circulating stream are isomerised to BPA under the prevailing conditions, so that the total yield of BPA can be increased.

For the further recovery of secondary components, the resin can also be subjected to thermal, acid- or base-catalyzed cleavage. The phenol released in this case, and optionally also isopropenylphenol, can be separated off by distillation and returned to the reaction.

The BPA-phenol adduct crystals obtained following the suspension crystallization of the reaction solution and solid-liquid separation described above are fed into further purification steps, wherein the separation of phenol and optionally the reduction of the concentration of secondary components are achieved. Thus, the BPA-phenol adduct crystals can be recrystallised for further purification from phenol, organic solvents, water or mixtures of the above solvents, which can optionally also contain BPA and its isomers, by a suspension crystallization. By selecting suitable solvents, the phenol present in the adduct crystals can also be completely or partly removed at the same time. Any phenol remaining in the BPA after the re crystallization is then separated off completely by suitable distillation, desorption or extraction methods.

Alternatively, the phenol can also be removed from the BPA-phenol adduct crystals by melting-out processes.

After separating off the phenol, a bisphenol A melt is obtained, which can be used for the production of polycarbonate by the transesterification process (melt polycarbonate) without previous solidification. However, the bisphenol A melt can also be solidified by known processes, e.g. by the prilling process or by flaking, for sale or further use. The melt can also be dissolved in sodium hydroxide solution and used for the production of polycarbonate by the interfacial polycondensation process. The bisphenol A that has been freed of phenol can optionally be subjected to another purification step, such as e.g. melt crystallization, distillation and/or recrystallization from phenol, water or an organic solvent, such as e.g. toluene, or mixtures of these substances, before further processing.

In the context of the process described, the content of secondary components, the so-called isomers, plays a decisive role in the quality of the bisphenol. These so-called isomers (indanes, chromanes, trisphenols, o,p-BPA etc.) influence the crystallization of the bisphenol A from the reaction solution. As their content in the reaction solution increases, their influence also grows. To achieve adequate quality in the crystallization in spite of this, parts of the circulating stream, the so-called BPA resin, have to be discharged from the circulation, as already described above. For economic reasons, it is necessary to keep the quantity discharged as small as possible since phenol and acetone are lost here as bisphenol A and isomers. While it is true that the processes known to the person skilled in the art, such as rearrangement and resin cleavage, make it possible to recover some of the raw materials, this is however associated with energy input and additional investment costs.

The object of the present invention was therefore to provide a process for the production of bisphenol A in which the isomer formation during the reaction is reduced and a high purity of bisphenol A in the end product is achieved after the crystallization and filtration, and thus the quantity discharged from the circulating stream, the so-called BPA resin, can be kept low.

### SUMMARY OF THE INVENTION

A process for the production of bisphenol A is disclosed. The process entails a) mixing phenol and acetone to form a mixture, and b) bringing the mixture to a temperature of 48 to 54° C. to form a warm mixture, and c) commencing a reaction between phenol and acetone upon contacting the warm mixture with an acid ion exchange catalyst to form a product mixture that contains bisphenol A. The bisphenol A may be extracted from the product mixture by crystallization and filtration.

### DETAILED DESCRIPTION OF THE INVENTION

It has now been found that this object can be achieved by a special way of conducting the reaction.

The invention relates to a process for the production of bisphenol A, wherein
a) phenol and acetone are mixed together, and
b) the mixture containing phenol and acetone is brought to a temperature in the range of 48 to 54° C., and then
c) the mixture containing phenol and acetone is brought into contact with an acid ion exchanger as catalyst at this temperature, and
d) the mixture containing phenol and acetone is reacted to form bisphenol A.

It is an essential feature of the process according to the invention that the mixture containing phenol and acetone is brought to a temperature of 48 to 54° C., preferably 50-53° C., particularly preferably 51.5 to 52.5° C. in step b) before the reaction.

The acid ion exchanger in step c) is preferably used in combination with a co-catalyst. These are usually thiols, which carry at least one SH function. The co-catalyst can be both homogeneously dissolved in the reaction solution and, in the case of the acid ion exchangers, fixed on the catalyst itself. Homogeneous co-catalysts are e.g. mercaptopropionic acid, hydrogen sulfide, alkyl sulfides, such as e.g. ethyl sulfide, and similar compounds. Fixed co-catalysts are aminoalkyl thiols and pyridylalkyl thiols which are ionically bonded to the catalyst, it being possible for the SH function to be protected and only released during or after fixing to the catalyst. The co-catalyst can also be covalently bonded to the catalyst as alkyl or aryl thiol.

Other substances may also be contained in the mixture containing phenol and acetone. For example, in addition to p,p-bisphenol A itself, the so-called isomers, which are contained in the recycled partial stream of the mother liquor, which originates from the crystallization and filtration of the BPA-phenol adduct, may also be contained therein. These are the compounds known to the person skilled in the art, such as e.g. o,p-bisphenol A, o,o-bisphenol A, trisphenols, (hydroxy-phenyl)chromanes, (hydroxyphenyl)indanes, (substituted) indanes, (substituted) indenols, (substituted) spirobisindanes, isopropenylphenol and its dimers and oligomers, (substituted) xanthenes, as well as other more highly condensed compounds with three or more phenyl rings in the molecular backbone. In addition, other substituted phenols, anisoles, methanol, mesityl oxide, mesitylene, diacetone alcohol and water, degradation products of the catalyst and of the co-catalyst as well as impurities from the raw materials may also be contained in the recycled partial stream.

By cooling the mixture containing phenol and acetone from the otherwise conventional 55 to 60° C. to 48 to 54° C., the starting temperature of the reaction is ultimately reduced to a temperature in the range of 48 to 54° C. As a result, the isomer formation during the reaction on the acid ion exchanger becomes more selective with regard to p,p-bisphenol A, the desired main product. At the same time, the quantity to be discharged from the recycled partial stream of the mother liquor, which originates from the crystallization and filtration of the BPA-phenol adduct crystals, i.e. ultimately the quantity of BPA resin to be discharged to keep the content of by-products, the so-called isomers, in the reactor constant at a level that is acceptable for the performance of the crystallization and the purity of the end product, is reduced. Because of the smaller discharge, less bisphenol resin is formed as a residual substance. Thus, the quantity of BPA resin is a direct indication of the isomer formation in the reaction. By reducing the reactor inlet temperature, the resin formation may be reduced by up to 50%, which represents a large economic saving with constant product quality.

The reaction is preferably conducted in such a way that a reactor temperature of 77° C. is not exceeded. It is preferable to conduct this reaction adiabatically. In practice, this generally leads to the highest temperature occurring at the reactor outlet. The reactor outlet temperature is therefore the highest temperature occurring in the reactor. Conducting the reaction adiabatically here also includes a method of conducting the reaction in which the reactor jacket is heated slightly from the outside to avoid crystallization in wall areas.

As a result of the low temperature at the beginning of the reaction, at which a high concentration of acetone is still present, in particular the self-condensation of acetone as well as the formation of chromanes, indanes and other by-products of bisphenol A production known to the person skilled in the art are reduced. To obtain a bisphenol of sufficient quality and to be able to perform the crystallization and filtration of the bisphenol A-phenol adduct crystals without any problems, a content of 100 g/l of the so-called isomers in the reaction mixture after the reaction should, if possible, not be exceeded. A content of 60 to 100 g/l of the so-called isomers in the reaction mixture at the reactor outlet is preferably established. As a result of the process according to the invention, the discharge of the partial stream of the recycled mother liquor originating from the crystallization and filtration of the BPA-phenol adduct crystals may be quantitatively reduced without exceeding the limit of 100 g/l of the so-called isomers in the product mixture at the reactor outlet. A process is therefore preferred in which a product mixture is obtained in step d) from which a bisphenol A-phenol adduct is then crystallized out and filtered off and bisphenol A produced therefrom, and wherein the mother liquor forming during the crystallization and filtration is partly recycled into the mixing of phenol and acetone in step a), wherein a partial stream is discharged from the recycled mother liquor and wherein this partial stream makes up quantitatively less than 6 wt. %, based on the quantity of bisphenol A produced, ignoring any phenol present. The discharged partial stream of the mother liquor therefore amounts to less than 6 wt. %, quantitatively, based on the quantity of bisphenol A produced, taking into account all the components contained in the partial stream except phenol. The content of phenol in the partial stream of the mother liquor to be discharged may readily be determined by the person skilled in the art using common analytical methods.

The quantity of BPA resin ultimately formed may be further reduced in the process according to the invention by methods known to the person skilled in the art, such as e.g. rearrangement and resin cleavage.

In particular the formation of indanes, indenols and spirobisindanes is favored by high temperatures. Formulae (I) and (II) show examples of indanes, formula (III) an example of an indenol and formula (IV) an example of a spirobisindane.

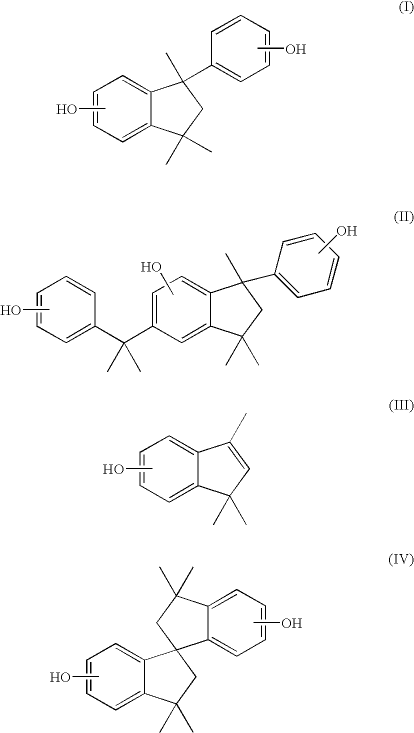

It is known that isomers such as o,p-BPA may still be rearranged during the reaction, whereas indanes, spirobisindanes and indenols cannot. Their formation in the reaction must therefore be especially avoided as far as possible and their concentration in the reaction mixture kept low.

It has been shown that the content of these indanes, spirobisindanes and indenols in the product mixture at the reactor outlet may be reduced by the process according to the invention to less than 15 g/l.

The process according to the invention enables a BPA to be produced in a purity of more than 99.5 wt. % of p,p-bisphenol A after crystallization and filtration of the BPA-phenol adduct, subsequent washing with phenol and separation of the phenol by distillation and/or desorption, without additional purification by recrystallization being necessary.

The bisphenol A produced by the process according to the invention may be reacted with phosgene by the interfacial polycondensation process or with diaryl carbonates, preferably diphenyl carbonate, by the melt process to form polycarbonate.

### EXAMPLE 1

### According to the Invention

A reaction solution consisting of 4 wt. % acetone, 6 wt. % isomers(indanes, chromanes, trisphenols, o,p-BPA etc.), 7 wt. % bisphenol A, 0.05 wt. % water, 300 ppm mercaptopropionic acid and the rest being phenol (about 83 wt. %) is passed from top to bottom through a reactor charged with 100 m³ of phenol-moist, acid ion exchanger Lewatit SC104 at a throughput of 30 t/h. This corresponds to a bisphenol A production of 4.2 t/h. The reactor inlet temperature is maintained at 52° C. The reactor outlet temperature is 75° C. At this setting, the partial stream of mother liquor discharged amounts to 5.1 wt. %, based on the weight of bisphenol A produced, taking into account all the components contained in the partial stream except phenol. The content of indanes, spirobisindanes and indenols is 12 g/l in total in the reactor discharge using this method of operation.

### EXAMPLE 2

### Comparative Example

The test is performed as in Example 1, but the reactor inlet temperature is now 56° C. and the reactor outlet temperature 79° C. At this setting, the partial stream of mother liquor discharged amounts to 8 wt. %, based on the weight of bisphenol A produced, taking into account all the components contained in the partial stream except phenol. The content of indanes, spirobisindanes and indenols is 19 g/l in total in the reactor discharge using this method of operation.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1. A process for the production of bisphenol A comprising
a) mixing phenol and acetone to form a mixture and
b) bringing the mixture to a temperature of 48 to 54° C. to form a warm mixture, and
c) commencing a reaction between phenol and acetone upon contacting the warm mixture with an acid ion exchange catalyst to form a product mixture that contains bisphenol A.

2. The process according to claim 1, wherein the acid ion exchange catalyst is used together with a co-catalyst.

3. The process according to claim 1 wherein the reaction takes place at a temperature not exceeding 77° C.

4. The process according to claim 2 wherein the reaction takes place at a temperature not exceeding 77° C.

5. The process according to claim 1 wherein the reaction is conducted adiabatically.

6. The process of claim 1 wherein the product mixture contains indanes, spirobisindanes and indenols in total amount of less than 15 g per liter of product mixture.

7. A process for the production of polycarbonate comprising
(i) preparing bisphenol A according to claim 1 and
(ii) reacting the bisphenol A with phosgene by the interfacial polycondensation to form polycarbonate.

8. A process for the production of polycarbonate comprising
(i) preparing bisphenol A according to claim 1 and
(ii) reacting the bisphenol A with diphenyl carbonate by the melt process to form polycarbonate.

9. Process for the production of polycarbonate, wherein bisphenol A is produced according to claim 1 and then reacted with phosgene by the interfacial polycondensation process or with diphenyl carbonate by the melt process to form polycarbonate.
